(19) United States
(12) Patent Application Publication
(10) Pub. No.: US 2002/0049386 A1
(43) Pub. Date: Apr. 25, 2002

(54) Multi-spectral fluorescence imaging and spectroscopy device

(76) Inventors: Victor X.D. Yang, Toronto (CA); Brian C. Wilson, Toronto (CA); Paul J. Muller, Toronto (CA); Peter R. Herman, Mississauga (CA)

Correspondence Address: DOWELL & DOWELL PC, SUITE 309, 1215 JEFFERSON DAVIS HIGHWAY, ARLINGTON, VA, 22202

(21) Appl. No.: 09/971,001

(22) Filed: Oct. 5, 2001

Related U.S. Application Data
(60) Provisional application No. 60/238,020, filed on Oct. 6, 2000.

(51) Int. Cl. A61B 6/00

(52) U.S. Cl. 600/476; 356/326

(57) ABSTRACT

The present invention provides a multi-spectral optical, open-field detection system utilizing light-induced-fluorescence imaging and point spectroscopy for use in in-situ diagnosis and treatment of diseased tissue with reduced sensitivity to changes in light source-target-detector geometry. The light excitation and detection optics share a common coaxial optical path which makes the present system very robust to changes in illumination-target-detector geometry. The apparatus may be used for the detection and localization of tumors or other pathological tissues both prior to treatment and for the detection of residual diseases during or after surgery or other treatments to remove or destroy the diseased tissue.

### CROSS REFERENCE TO RELATED UNITED STATES PATENT APPLICATION

This patent application relates to U.S. Provisional patent application Serial No. 601 238, 020 filed on Oct. 6, 2000, entitled MULTI-SPECTRAL FLUORESCENCE IMAGING AND SPECTROSCOPY DEVICE.

### FIELD OF THE INVENTION

This invention relates to methods and devices relating to open-field light-induced-fluorescence imaging and spectroscopy for diagnosis and treatment of solid tissue disorders.

### BACKGROUND OF INVENTION

Substantial research has been and is being carried out on tumor detection utilizing light-induced fluorescence imaging and spectroscopy. In such measurements, a light source of a certain wavelength or wavelengths is used to excite the target tissue, and the intrinsic fluorescence emitted by the tissue, or autofluorescence signal, is detected. The autofluorescence signal usually spans the entire visible/near Infrared spectrum. Specific fluorescent drugs, such as photosensitizers or tumor markers, can be injected into the patient prior to the measurements, in which case the extrinsic fluorescence, or drug-induced fluorescence signal is measured. The drug induced fluorescence emission signal usually occupies a smaller optical bandwidth than the autofluorescence. Both spectroscopic and imaging detectors can be used in such measurements. The results suggest that if artefacts due to source-target-detector variations, excitation illumination intensity variations, and optical collection efficiency variations are minimized, the combined Information delivered by both the imaging and spectroscopy can be used to distinguish normal from diseased tissue, and further to separate diseased tissue into different stages of the tumor development. See, e,g., Yang V et al, “Non-Contact Point Spectroscopy Guided by Two-Channel Fluorescence Imaging in a Hamster Cheek Pouch Model”, Proc. SPIE Vol: 3592, 1999, and Kluftinger A et at, “Detection of Squamous Cell Cancer and Pre-Cancerous Lesions by Imaging of Tissue Autofluorescence In the Hamster Cheek Pouch Model”, Surgical Oncology, 1: 183-188,1992.

The use of photodynamic therapy to treat various forms of cancer has received considerable attention in the past two decades. The research and development in the areas of photosensitizers, appropriate light sources, and delivery methods have produced several generations of photosensitizer, e.g., Photofrin, Aminolevulinic acid,, etc., different type of light sources, e.g., arc lamps, dye lasers, laser diodes, etc., as well as fiber optic and endoscopic light delivery systems. For certain types of cancer, such as brain tumor, the benefit of photodynamic therapy is substantial, see for example Muller P. J. and Wilson B. C., “Photodynamic Therapy of Malignant Brain Tumors”, Lasers in Medical Science”, 5:245-252, 1990; and Muller P. J. et al., “Multivariate Analysis of Primary Brain Tumors Treated with Photodynamic Therapy”, Canadian Journal of Neurol Sciences 19:2Q56, 1992. The treatment light source can be site directed after the specific location and extent of the tumor tissue is pinpointed by methods outlined in (1).

Optical monitoring of the photodynamic therapy treatment is also possible, either by utilizing photosensitizer fluorescence or absorption, or tissue reflection. These can be used to monitor the changes occurring to the photosensitzer or to the tissue during the photodynamic therapy and providing information for the treatment dosimetry, see for example Potter W R, “PDT Dosimetry and Response”, in: Dougherty T J (ed) Photodynamic Therapy: Mechanisms, Proc. SPIE, 1989, 1065:88-99; and Wilson B C et al, “Implicit and Explicit Dosimetry in Photodynamic Therapy: a New Paradigm”, Lasers in Medical Science, 1997, 12:182-199. In general, the optical signals for monitoring span across the entire visible/near infrared spectrum. The information derived can then be used to modulate the treatment, in terms of changing the excitation light beam intensity, duration, fractionation, and location.

U.S. Pat. No. 5,345,941 issued to Rava et at. is directed to a method of contour mapping of spectral diagnostics using laser induced fluorescence of tissue. U.S. Pat. No. 5,419,323 issued to Kittrell et al. is directed to a method of laser induced fluorescence of tissue wherein an optical fiber delivers light to the tissue sample. U.S. Pat. No. 5,833,617 issued to Hayashi discloses a fluorescence detecting apparatus for studying living tissue using two spectral windows. U.S. Pat. No. 5,999,844 issued to Gombrich et al. teaches a method of imaging and sampling diseased tissue using autofluorescence with the optical system mounted in an endoscope. Similarly, U.S. Pat. No. 5,413,108 (Alfano), U.S. Pat. No. 5,971,918 (Zanger) and U.S. Pat. No. 5,590,660 (MacAulay et al.) disclose endoscopic based devices for tissue analysis using fluorescence. U.S. Pat. No. 5,421,337 issued to Richards-Kortum et al. discloses a method of spectral diagnosis of diseased tissue. U.S. Pat. No. 5,665,754 issued to Sevick-Muraca et al. discloses a method of fluorescence imaging of biological tissue.

U.S. Pat. No. 5,699,798 issued to Hochman discloses a system for fluorescence imaging and detection of malignant gliomas during open field neurosurgery using a modified surgical microscope to achieve fluorescence imaging. Surgical microscopes are optimized for white light microscopy, and are not optimized for fluorescence imaging, which requires high optical throughput. Disadvantages to this system include the fact that it is very sensitive to changes in light source-target-detector geometry and it is not per se multi-spectral in that it is essentially a two-channel imaging device, In addition, high-resolution spectroscopic measurement is not possible with Hochman's apparatus due to the lack of a spectrograph in the apparatus.

Therefore, optical-based diagnosis, photodynamic-based treatment, and monitoring of solid tissue disorders, including cancer, requires an instrument which is deployable in a clinical setting, such as a surgical environment, and which can distinguish diseased tissue from normal tissue, and monitor the treatment process. It would be useful if all of these functions could be carried out optically, in a non-invasive manner without physically contacting the patient.

In order to carry out all of these functions within the visible/near infrared spectrum, multi-spectral methods should be utilized. Specifically, due to the different information content of signals, i.e., some contain spatial information of the tumor cell position, some contain spectral information of the components of the tissue bio-molecules, imaging and spectroscopic sensors must be utilized. In addition, this information has to be conveyed in a manner such that it can be synergistically overlaid onto the existing knowledge of the user.

Therefore, it would be very advantageous to provide a multi-spectral, open-field detection apparatus utilizing light-induced-fluorescence imaging and spectroscopy for use in in-situ diagnosis and treatment with reduced sensitivity to changes in light source-target-detector geometry.

### SUMMARY OF THE INVENTION

The invention provides a method and device for detecting diseased tissue in a patient, guiding therapeutic treatment, and monitoring the therapeutic treatment.

In one aspect of the invention there is provided an optical device for illuminating a target area and detecting electromagnetic radiation reflected or emitted by fluorescence from said target area, comprising:

a light source and filter means for transmitting selected wavelengths of electromagnetic radiation emitted from said light source;

a housing including first optical focusing means for shaping and directing a first light beam containing said selected wavelengths along a first optical path and focusing said light beam onto a selected surface area of a target area located a selected distance from said housing;

a detection means for detecting light; and

said housing Including second optical focusing means for shaping and directing electromagnetic radiation reflected or emitted from said target area as a second light beam along a second optical path and focusing said electromagnetic radiation onto said detection means, wherein said first optical path and said second optical path share a coaxial optical path section.

In another aspect of the invention there is provided a multi-spectral fluorescence imaging and spectroscopic apparatus for fluorescence imaging and spectroscopy, comprising:

a light source and filter means for transmitting selected wavelengths of electromagnetic radiation emitted from said light source;

a housing and positioning means for positioning said housing in preselected positions, said housing including a first optical focusing means for shaping and directing a first light beam containing said selected wavelengths along a first optical path and focusing said light beam onto a selected surface area of a target area located a selected distance from said housing;

detection means including a first light detector and a spectrometer; and

said housing including second optical focusing means for shaping and directing electromagnetic radiation reflected or emitted from said target area as a second light beam along a second optical path and focusing said electromagnetic radiation onto said detection means, wherein said first optical path and said second optical path share a coaxial optical path section, said second optical focusing means including means for splitting said second light beam into a third light beam and directing said third light beam toward said spectrometer and a fourth light beam directed toward, and imaged onto, said first light detector for producing an image of the target area from which light is diffusely reflected and/or emitted by fluorescence.

The present invention also provides a method for illuminating a target area and detecting electromagnetic radiation reflected or emitted by fluorescence from the target area, comprising the steps of:

shaping and directing a first light beam containing selected wavelengths along a first optical path and focusing said light beam onto a selected surface area of a target area; and

shaping and directing electromagnetic radiation reflected or emitted from said target area as a second light beam along a second optical path and focusing said electromagnetic radiation as an image onto a light detection means, wherein said first optical path and said second optical path share a common coaxial optical path section so that only the region of the target area where diffusely reflected light or light emitted by fluorescence are being detected is being illuminated by said first light beam; and

d) processing signals output from said light detection means and displaying the processed signals in a selected format.

In this aspect of the invention the target area may be human tissue such as a human brain of a patient undergoing in-situ diagnosis and treatment of diseased tissue utilizing light-induced-fluorescence imaging and point spectroscopy.

The step of processing signals output from the spectrometer and the first light detector may include displaying images and spectra produced therefrom in such a way that highlights optical differences between target tissue and/or surrounding tissue.

The method may include illuminating the tissue with a multispectral white-light beam, and including imaging of reflected light and fluorescence imaging of normal and tumorous brain tissue during neurosurgery.

### BRIEF DESCRIPTION OF THE DRAWINGS

The invention will now be described, by way of non-limiting examples only, reference being had to the accompanying drawings, in which:

FIG. 1 Is a block diagram of a fluorescence multi-spectral imaging apparatus constructed in accordance with the present invention;

FIG. 2 is a schematic diagram of the optical layout of the fluorescence multi-spectral Imaging apparatus of FIG. 1;

FIG. 3 is a schematic block diagram of the Interfacing between the hardware and software components of the multifluorescence imaging system,,

FIG. 4 shows a schematic cross section of a surgical field with tumors present;

FIG. 5 is a fluorescence image of a resolution target;

FIG. 6 shows the fluorescence intensity line profile of the pattern groups 0, 1, and 2, as indicated in FIG. 5;

FIG. 7 shows the modulation transfer function (MTF) of the imaging system of the present invention;

FIG. 8 shows the intensity line profile of a reflection test target sampled by the non-contact point spectroscopy;

FIG. 9 shows the peak fluorescence intensity of an optical brain phantom, as a function of the concentration of a fluorescent marker;

FIG. 10 shows the 630 nm peak fluorescence intensity captured by non-contact point spectroscopy from an optical brain phantom, as a function of the concentration of a fluorescent marker.

### DETAILED DESCRIPTION OF THE INVENTION

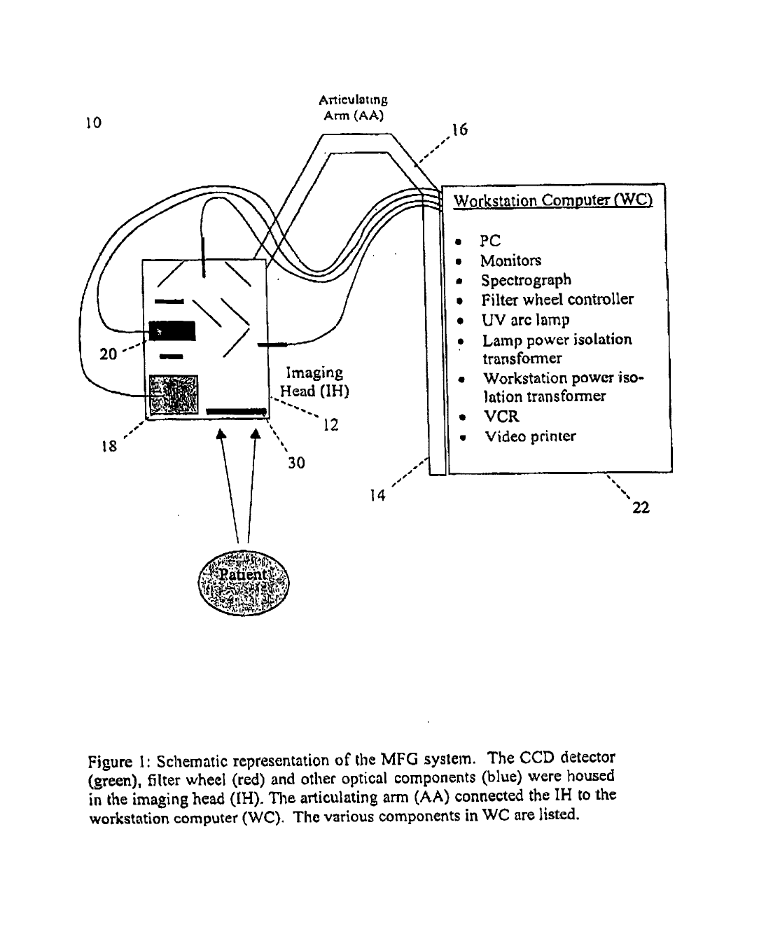

Referring first to FIG. 1, a multi-spectral fluorescence guidance imaging and spectroscopy apparatus is shown generally at 10. The apparatus includes an imaging head 12 attached to a workstation support frame 14 by a positioning member 16 so that head 12 can be positioned in selected positions and orientations with respect to the object being illuminated. The imaging head 12 houses a 2-D array optical detector such as a charge coupled device (CCD) detector 18 (but other 2-D optical detectors may be used) and a filter wheel 20. The various instruments making up the controller 22 are located on support frame 14 and are optically and electronically coupled to the appropriate components of the imaging head 12. In one embodiment the positioning member for positioning the imaging head 12 is an articulated supporting arm 16 with four joints offering six degrees of freedom, which supports imaging head or housing 12 and carries the electrical/optical connections between head 12 and controller 22. The articulating arm 16 may be under the control of controller 22 to allow it to be positioned or repositioned as needed during the various procedures.

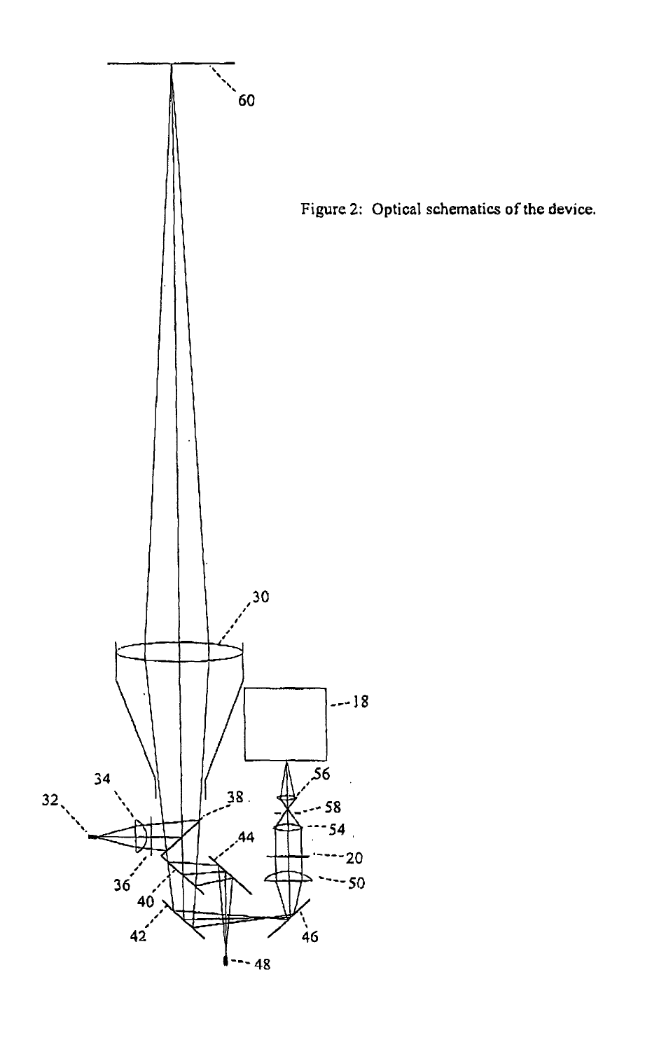

The optical layout of the multi-spectral fluorescence guidance imaging system is shown in greater detail in FIG. 2. It will be appreciated that the embodiment of the optical system shown in FIG. 2 is exemplary in nature only and is not intended to limit the present Invention. For example the values for focal lengths (f), diameters (D) and the like of the various components may be varied as desired. The optical layout of the fluorescence multispectral imaging device includes a primary objective lens 30 having a long focal length (f=235 mm, D=120 mm) with an anti-reflection coating for the visible/near infrared spectrum, a liquid light guide 32, a plano-convex lens 34 (f=38 mm, D=25.4 mm), an excitation filter 36 (D=25.4 mm, λₚₐₛₛ=405±15 nm), dichroic long-pass filter 38 (D=50.8 mm, λ_{cutoff}=475 nm), a 50-50% beam splitter 40 (D=25.4 mm); three planar mirrors 42, 44 and 46 (D=50.8 mm), an optical fiber coupler 48 mounted on a x-y-z translational stage and optically coupled to the spectrograph located on the work-station, a plano-convex lens 50 (f=100 mm, D=50.8 mm), filter wheel 20, lenses 54 and 56 with an iris 58 with manual iris adjustment, and the CCD detector (camera) 18. Filter wheel 20 is preferably a motorized filter-wheel mounted with several band-pass filters for passing pre-selected wavelengths in the visible/near infrared spectrum. In one embodiment a thermal electrically cooled CCD detector was used.

The optical fiber coupler 48 is mounted on the x-y-z translational stage with the relative positions of the input end of the fiber (into which light is coupled and the camera 18) such that the input end of the fiber is in the center of the field of view of the images on camera 18. By adjustment in the z-direction, light from a point source at the center of the image would be focused into the fiber.

The computerized controller 22 includes a computer processor designed around an Intel-based computer system, containing a high-speed digital image acquisition card; a spectrographic signal acquisition card connected to the spectrograph; and high volume data storage disks. The controller 22 includes a filter wheel controller for filter wheel 20 on head 12, the UV lamp source and lamp power supply, a VCR and video printer. More particularly, in order to acquire a digital image, a 16-bit image acquisition card was used and, because this is a bus-mastering device, no CPU intervention is required during the image acquisition, thereby freeing the main computer to perform data processing. A graphics card with 4 MB of RAM was used to display the digital images. A secondary analog television monitor displayed the NTSC output of the camera. The computer algorithm for controlling the user interface and data display is based on an object oriented graphical instrumentation language.

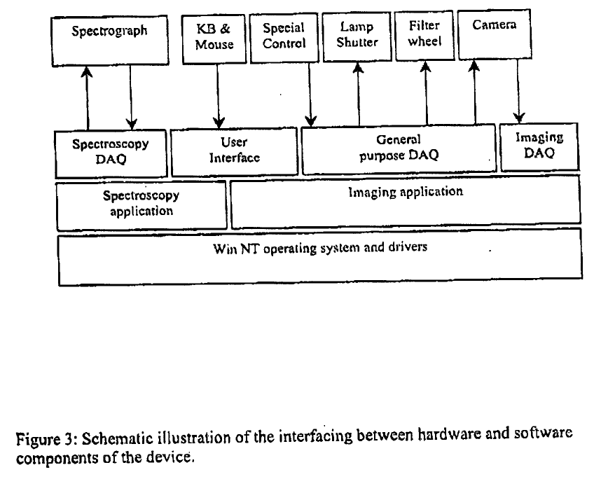

A schematic block diagram of the interfacing between the hardware and software components of the multi-fluorescence imaging system is shown in FIG. 3. Running on top of the Windows NT operating system are two application programs. The spectroscopy program communicates with the data acquisition card (DAQ) for the spectrograph, which In turn communicates with the spectrograph. The imaging program communicates with two DAQ cards. The general purpose DAQ card controls the arc lamp shutter and the filter wheel, while the imaging DAQ controls the camera and acquired images. Emergency shutdown switches, one for shutting off power to the arc lamp and the other for shutting all power to the optical system as a whole constitute the special control box.

The visible/near infrared spectrograph is a miniaturized device with a holographic grating for the visible/near infrared wavelength band, a set of input/output slits, and a photodiode array detector. As mentioned above, the spectrograph acquires the signals from a variable area and/or location of the target tissue, selected via a joystick controlled motor-positioning system, simultaneously and/or sequentially with respect to the imaging process. In this particular embodiment, the 2-D array optical detector and the spectrometer were chosen to be sensitive in (but not limited to) the visible and near infrared wavelength range.

In operation, the liquid light guide 32 receives light from a UV lamp source (e.g. mercury lamp) that emits in the violet-blue range including 405 nm and 436 nm. The liquid light guide delivers UV to the imaging head optics and in one embodiment the liquid light guide was 3 meters long and 5 mm in diameter and provided about 800 W/cm² of output power. The light beam Is focused by piano-convex lens 34 through the interference band-pass filter 36 which passes light of wavelength λₚₐₛₛ=405±15 nm. The 405 nm light is incident at 45° on the dichroic long pass filter 38 which reflects light of wavelength λ<λ_{cutoff} with a reflectance of R=97% and transmits wavelength λ>λ_{cutoff} with a transmittance T=85% so that the 405 nm light is reflected toward objective lens 30 and subsequently focused by lens 30 onto a focal plane located at 60.

The light reflected from the object at the focal plane 60 and fluorescence emitted by the object are collected by the lens 30 and passed back through the dichroic long pass filter 38 and then through the 50/50% beam splitter 40 which divides the light for imaging and spectroscopy purposes. The portion reflected by beam splitter 40 is reflected off mirror 44 and focused down into the optical fiber 48 which is a 400 nm fiber. The point spectroscopy is obtained by positioning the input end of fiber 48 at the first image plane following the main object lens 30. The fiber 48 then delivers this light to the spectrometer. The input end of fiber 48 may be positioned in the center of the image or alternatively its position can be adjusted using the x-y translational stage to enable the operator to record spectra from different locations on the image, selected by for example a hand-manipulated device such as a joystick.

The various optical elements (including for example lenses, beam splitters, mirrors, adjustable apertures, filters) for shaping and focusing the light beam directed onto the target surface and for shaping and focusing the light reflected, or emitted by fluorescence, from the target onto the light detector from which an image is produced or onto the fiber leading to the spectrometer, can be mounted on adjustable mounts which can be connected to the computer for adjustment of beam sizes, focal lengths, beam intensity of both beams.

The optical design of this particular embodiment permits the achievement of extremely small sampling volumes (less than one part per million of a liter) at a large working distance (greater than 50 cm). This ability to sample small volumes of tissue is important for tumor boundary identification and differentiation between tumor or normal tissue. The ability to perform such identification and differentiation at a large working distance is also important for surgical applications where surgeon's accessibility to the target tissue can not be compromised.

In practice, when the point spectroscopy being recorded is from turbid (light scattering) tissue, the effective sampling area, or more precisely, the effective sampling volume depends on the optical properties of the tissue, as well as the depth of the source. However, a sampling diameter on the order of millimeters would be considered adequate during surgical procedures.

The portion of the light transmitted by beam splitter 40 undergoes multiple reflections by mirrors 42 and 46 and is then collimated by plano-convex lens 50 through one of the filters on filter wheel 20 whereupon it is focused by lenses 54 and 56 through iris 58 onto thermal-electrically (TE) cooled CCD detector 18 from which the image is formed and displayed on a CRT or video screen.

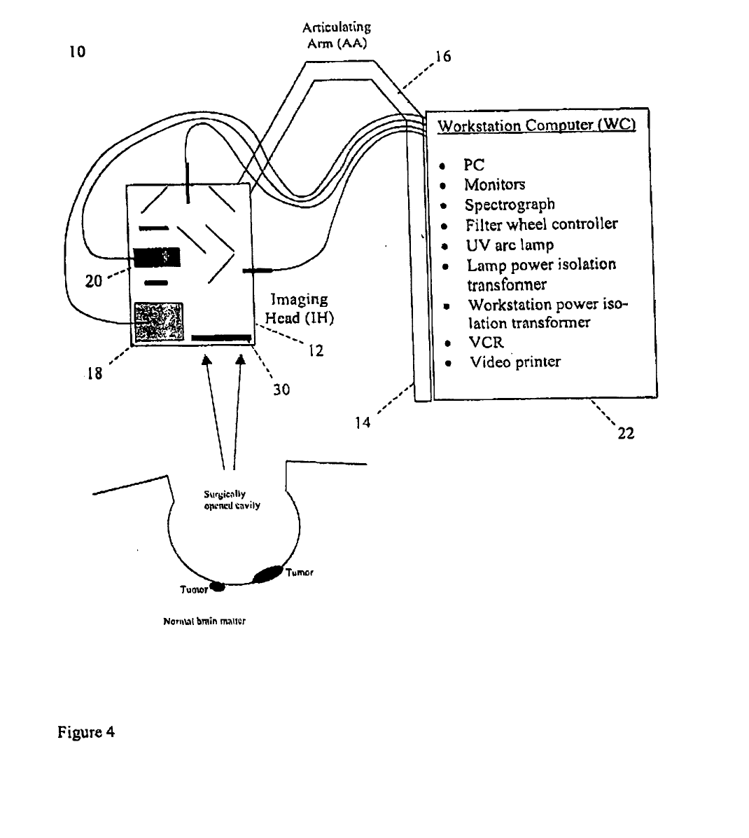

As can be seen from FIG. 2, the excitation light beam after it is reflected off long pass filter 38 is coaxial with the light signals reflected from the tissue sample at the excitation wavelength and fluorescent light signals emitted by the fluorescent species located in the tissue sample without interference between the various light signals. Thus the excitation light beam, and the returning light signals, whether diffusely reflected light or fluorescent light, are coaxial between the imaging head 12 and the sample under study which makes the present system very robust to changes in illumination-target-detector geometry. In non-coaxial excitation/detection systems, variations in the illumination-target-detector geometry not only cause changes in the illumination intensity, but also create lateral shifts in the illumination pattern as well as shadowing. In addition, a coaxial system Illuminates only the region where fluorescence images are being taken, thus reduce the target exposure of illumination light, which could photobleach the target and reduce the fluorescence intensity. Finally, the coaxial system disclosed herein has complete overlap of the illumination and detection light cones and so avoids the edge-clipping effects present in non-coaxial systems due to mismatch between the illumination and imaging cones in the optical path. For example, this is important in neurosurgical resection of brain cancer, where the surgical fields (see FIG. 4) often have complex geometries in which clipping can occur.

The multi-spectral fluorescence guidance imaging system disclosed herein is very advantageous over previous systems in that it provides multi-spectral, non-contact fluorescence and diffuse reflectance Imaging of a tissue surface in vivo.

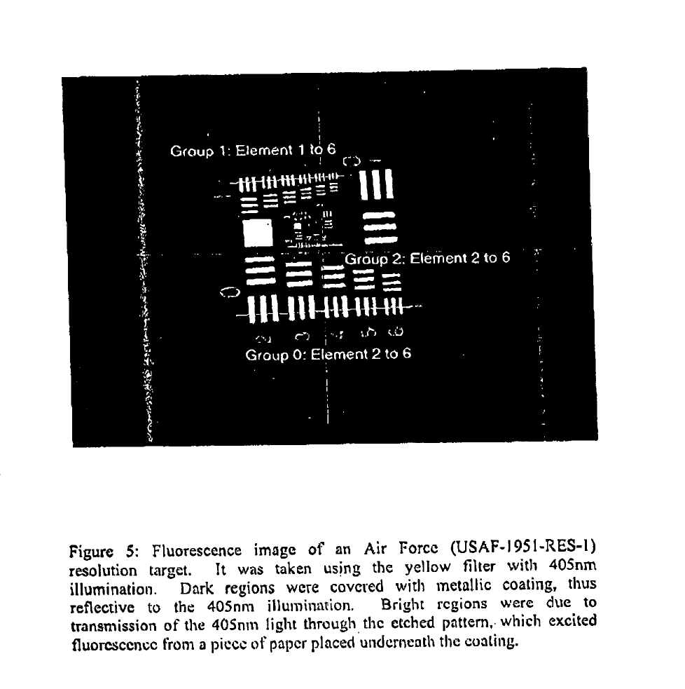

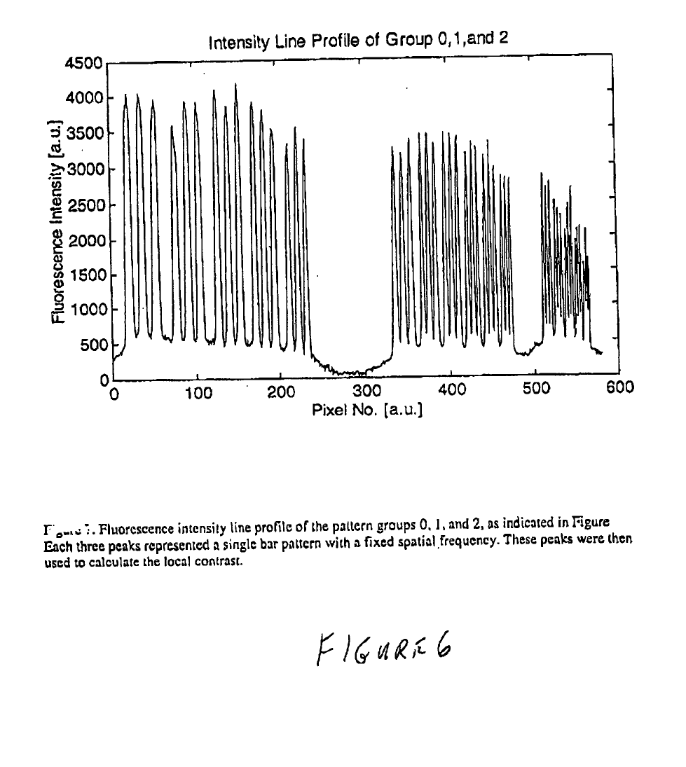

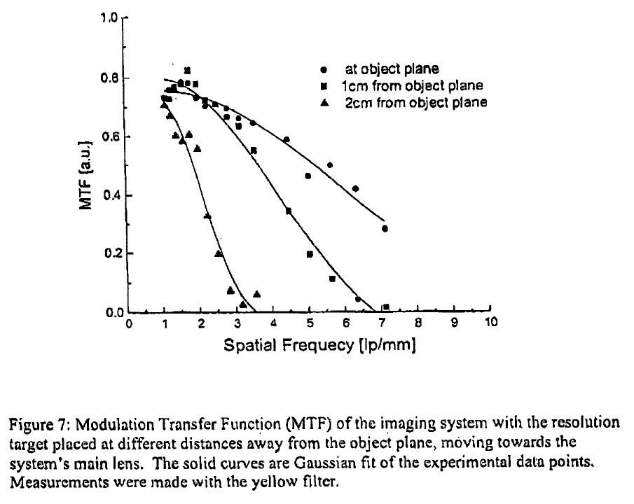

FIG. 5 shows the system's fluorescence imaging capability. The test target is a US AIR FORCE resolution target, which is an etched pattern on metallic coating on a transparent glass substrate. A piece of paper is placed below the coating to provide the fluorescence, Emitted by the imaging head, a beam of 405 nm illumination light is used to excite the fluorescence. The image shows the excellent spatial resolution of the device's imaging capability. To quantify this capability, FIG. 6 shows the measured fluorescence intensity along the resolution target, in which each set of three peaks represents a single bar pattern with a fixed spatial frequency, these peaks were then used to calculate the local contrast. From these measurements, the modulation transfer function (MTF) of the imaging system of the present Invention is calculated and plotted in FIG. 7. The MTF is measured with the resolution target placed at different distances away from the object plane and moving towards the system's main lens. The solid curves are Gaussian fit of the experimental data points, showing the measured contrast at different spatial resolution or frequencies.

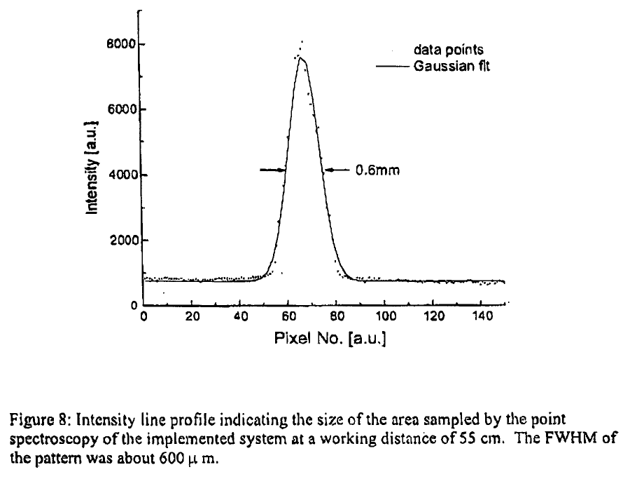

The non-contact point spectroscopy aspect of the present device has excellent spatial localization, as demonstrated by the small spot size of interrogation in FIG. 8. The Full Width at Half Maximum (FWHM) of the curve indicating the size of the area sampled by the non-contact point spectroscopy of the implemented system at a working distance of 55 cm, in which the FWHM of the pattern was about 0.6 mm. When taking point spectroscopy from turbid tissue, the effective sampling area, or more precisely the effective sampling volume depended on the optical property of the tissue, as well as the depth of the source. A sampling diameter of the order of millimeters is very useful during a surgical procedure.

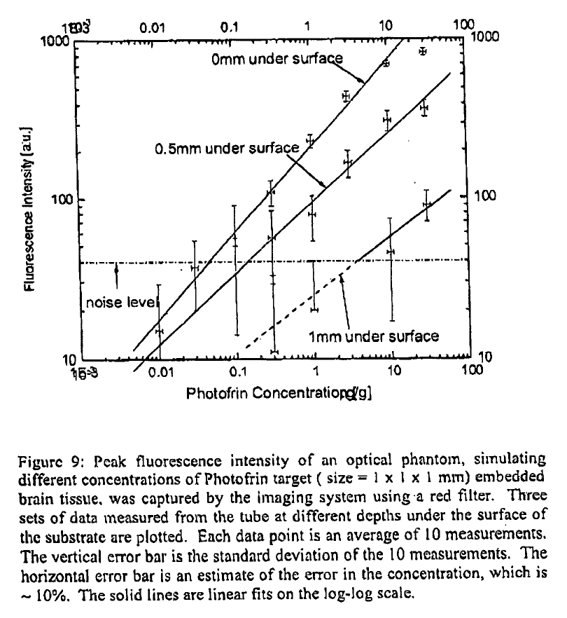

The sensitivity of the apparatus disclosed herein has been demonstrated to be able to detect very small amount of fluorophores. For example, FIG. 9 shows the peak fluorescence intensity detected from an optical phantom, which simulated different concentrations of Photofrin target (size−1×1×1 mm) embedded brain tissue. This was obtained from the multispectral imager. Three sets of data measured from the target at different depths under the surface of the phantom are plotted. Each data point is an average of 10 measurements. The vertical error bar is the standard deviation of the 10 measurements. The horizontal error bar is an estimate of the error in the concentration, which is about 10%. The solid lines are linear fits on the log-log scale. This shows the minimum concentration of Photofrin detected by the multispectral imager is less than 100 parts per trillion (ppt) of brain tissue, demonstrating the high sensitivity of the imager.

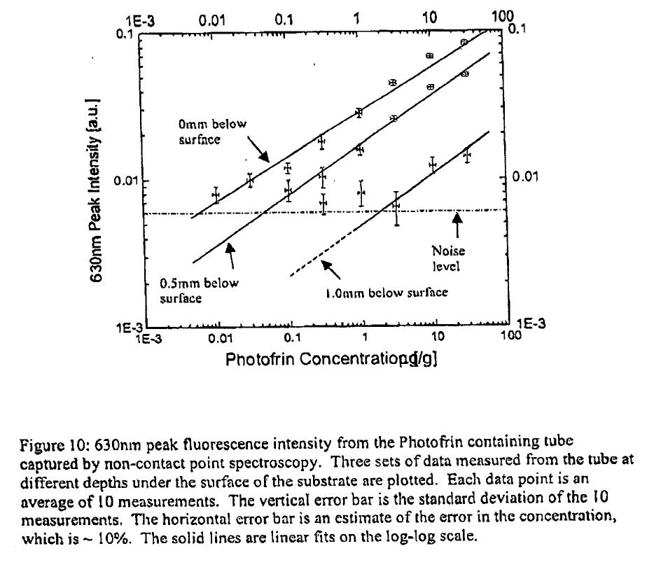

The non-contact point spectroscopy portion of the invented device has even higher sensitivity. FIG. 10 shows the 630 nm peak fluorescence intensity from the Photofrin target captured by non-contact point spectroscopy. Three sets of data measured from the tube at different depths under the surface of the phantom are plotted. Each data point is an average of 10 measurements. The vertical error bar is the standard deviation of the 10 measurements. The horizontal error bar is an estimate of the error in the concentration, which is about 10%. The solid lines are linear fits on the log-log scale. This shows the minimum concentration of Photofrin detected by non-contact point spectroscopy is less than 10 parts per trillion (ppt) of brain tissue, demonstrating the very high sensitivity of the non-contact point spectroscopy of the invented device.

The main novelty of this invention is thus four parts. First, the coaxial optical design allows complete overlap of the illumination and detection light cones, which reduces artefacts induced by illumination-target-detection geometry. Secondly, the multi-spectral aspects of the imager allow precise interrogation of the target and provide more information than single or dual spectral imaging devices. Third, the point non-contact spectroscopy provides a full spectrum, which allows much finer spectral resolution interrogation at any single point in the field of view than the imager. Finally, the coaxial design also allows precise spatial correlation between the point spectroscopy and the multispectral imaging. The benefits from these four parts are synergistic,

Various uses of apparatus 10 in operation will be described and it will be appreciated that these are non-limiting and only exemplary in nature. Under conventional lighting conditions, e.g., surgical lamp illumination, reflectance images are obtained of the target area of the patient within multiple specific spectral windows. The information is digitally processed, displayed, and stored. The target area of the patient is then irradiated using apparatus 10 with a beam of light of specific wavelength(s) and specific spatial distribution, such that one or more specific endogenous or exogenous fluorophore(s) within the tissue can be excited. The visible and/or near infrared fluorescence emission from such fluorophore(s) Is detected by detector 18 in a non-contact manner. The fluorescence emission signal is measured as a function of position (imaging), as a function of wavelength (spectroscopy), and as a function of both position and wavelength (multiple spectrally resolved imaging). The information is digitally processed, displayed, and stored. These results are then Integrated with the images taken under conventional lighting conditions and correlated with observed anatomical structures and landmarks. These measurements can be repeated and the information can be further processed as a function of time, which derives more information.

The information from the above types of measurements can then be further processed and integrated with other medically relevant information, such as CT and MRI scans of the patient. The combination of information from all these sources serve as the basis of the diagnostic, therapeutic guidance, and monitoring functions of the device in the invention. This information combined with additional information, such as drug dosage, can be further processed to provide information for photodynamic therapy dosimetry including, for example, measurement of photosensitizer uptake, distribution, and photobleaching.

It will be appreciated by those skilled in the art that although the present invention is very advantageous when used in conjunction with photodynamic therapy (PDT), it is certainly not necessarily to be restricted to this application alone. The open-field fluorescence imaging and spectroscopy apparatus disclosed herein may be used for imaging fluorescence tissue where the tissue surface is accessible before, during, and after surgery or other treatments, with or without the use of exogenous fluorescence drug; before, during, and after photodynamic treatment (PDT) and may use tissue auto-fluorescence and/or a exogenous fluorescence drug; before, during, and after any combination of surgery or other treatments and PDT and again may use tissue auto-fluorescence and/or an exogenous fluorescence drug. The apparatus may be used for the detection and localization of tumors or other pathological tissues prior to treatment,for guidance of surgery, photodynamic therapy or other treatments during treatment, and for the detection of residual diseases after surgery or other treatments to remove, destroy or otherwise modify the diseased or normal tissues.

The multispectral imaging and spectroscopy apparatus disclosed herein has been used for 1) multispectral white-light reflectance and fluorescence imaging of normal and tumorous brain tissue during neurosurgery; 2) multispectral whitelight reflectance and fluorescence imaging of normal and tumorous brain tissue before, during, and after photodynamic therapy; and 3) multispectral white-light reflectance and fluorescence imaging of normal and tumorous oral tissue. In all of the above applications, both autofluorescence and Photofrin fluorescence measurements have been used to obtain images.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

Therefore what is claimed is:

1. An optical device for illuminating a target area and detecting electromagnetic radiation reflected or emitted by fluorescence from said target area, comprising:
a light source and filter means for transmitting selected wavelengths of electromagnetic radiation emitted from said light source;
a housing including first optical focusing means for shaping and directing a first light beam containing said selected wavelengths along a first optical path and focusing said light beam onto a selected surface area of a target area located a selected distance from said housing;
a detection means for detecting light; and
said housing including second optical focusing means for shaping and directing electromagnetic radiation reflected or emitted from said target area as a second light beam along a second optical path and focusing said electromagnetic radiation onto said detection means, wherein said first optical path and said second optical path share a coaxial optical path section.

2. The device according to claim 1 including a data acquisition means connected to said light detection means, and including computer control means connected to said data acquisition means for controlling said light detection means and processing light signals detected by said light detection means.

3. The optical device according to claim 1 wherein said detection means includes a first light detector and a spectrometer, and wherein said means for shaping and directing electromagnetic radiation reflected or emitted from said target area as a second light beam along a second optical path includes means for splitting said second light beam into a third light beam and directing said third light beam toward said spectrometer and a fourth light beam directed toward and imaged onto said first light detection means thereby producing an image of the target area from which light is diffusely reflected and/or emitted by fluorescence.

4. The optical device according to claim 3 wherein said second optical focusing means includes selectable light filter means for transmitting selected wavelengths of said light beam being Imaged onto said first light detector.

5. The optical device according to claim 3 wherein said second optical focusing means Includes an optical fiber having a first end connected to said spectrometer and a second end located on a fiber mounting means located in said housing in a selected position so that said third light beam is focused as an image onto said second end of said optical fiber.

6. The optical device according to claim 5 wherein said fiber mount means includes adjustment means for adjusting a position of the second end of the fiber in x, y and z directions.

7. The optical device according to claim 3 including positioning means attached to said housing for positioning said housing in selected positions with respect to a target area to be illuminated.

8. The optical device according to claim 7 wherein said positioning means is an articulating arm with said housing mounted on one end of said articulating arm and the other end of the articulating arm attached to a support frame, said articulating arm providing six degrees of freedom to said housing.

9. The device according to claim 7 including first data acquisition means connected to said first light detector, a second data acquisition means connected to said spectrometer, and Including computer control means connected to said first and second data acquisition means and to said positioning means for controlling said first light detector and said spectrometer and processing light signals detected by said first light detector and said spectrometer.

10. The optical device according to claim 9 wherein said computer control means includes display means for displaying images from light detected by said first light detector and wavelength dependent spectra from light detected by said spectrometer.

11. The optical device according to claim 10 wherein said first light detector is one of a charge coupled device (CCD) detector, an array detector, and a film detector.

12. The optical device according to claim 2 wherein said computer control means includes visual display means for displaying images of data processed from light detected by said detection means.

13. The optical device according to claim 6 wherein said adjustment means is connected to an operator controlled actuator for operator controlled adjustment of the second end of said fiber within the image of the target area to enable the operator to record spectra from different locations on the image.

14. The optical device according to claim 12 wherein said operator controlled actuator is a hand-operated manipulator.

15. The device according to claim 13 including first data acquisition means connected to said first light detector, including second data acquisition means connected to said spectrometer, and including computer control means connected to said first and second data acquisition means and to said positioning means for controlling said first light detector and said spectrometer and processing light signals detected by said first light detector and said spectrometer.

16. The optical device according to claim 5 wherein said computer control means includes visual display means for displaying images from light detected by said first light detector and wavelength dependent spectra from light detected by said spectrometer.

17. The optical device according to claim 15 wherein said second optical focusing means includes selectable light filter means for transmitting selected wavelengths of said light beam being imaged onto said firstlight detector, said selectable light filter means includes a plurality of light filter in a motorized carousel, said motorized carousel being connected to said computer control means.

18. The optical device according to claim 15 wherein said first optical focusing means for shaping and directing said first light beam containing said selected wavelengths along said first optical path include optical components mounted on adjustable mounts which are controlled by said computer control means for computer controlled adjustment of focussing of said first light beam on said target,

19. The optical device according to claim 15 wherein said second optical focusing means for shaping and directing electromagnetic radiation reflected or emitted from said target area as a second light beam along said second optical path and focusing said electromagnetic radiation onto said detection means include optical components mounted on adjustable mounts which are controlled by said computer control means for computer controlled adjustment of focussing of said second light beam on said first light detector.

20. A multi-spectral fluorescence imaging and spectroscopic apparatus for fluorescence imaging and spectroscopy, comprising:
a light source and filter means for transmitting selected wavelengths of electromagnetic radiation emitted from said light source,
a housing and positioning means for positioning said housing in preselected positions, said housing including a first optical focusing means for shaping and directing a first light beam containing said selected wavelengths along a first optical path and focusing said light beam onto a selected surface area of a target area located a selected distance from said housing;
detection means including a first light detector and a spectrometer; and
said housing including second optical focusing means for shaping and directing electromagnetic radiation reflected or emitted from said target area as a second light beam along a second optical path and focusing said electromagnetic radiation onto said detection means, wherein said first optical path and said second optical path share a coaxial optical path section, said second optical focussing means including means for splitting said second light beam into a third light beam and directing said third light beam toward said spectrometer and a fourth light beam directed toward, and imaged onto, said first light detector for producing an image of the target area from which light is diffusely reflected and/or emitted by fluorescence.

21. The device according to claim 20 including first data acquisition means connected to said first light detector, including second data acquisition means connected to said spectrometer, and including computer control means connected to said first and second data acquisition means and to said positioning means for controlling said first light detector and said spectrometer and processing light signals detected by said first light detector and said spectrometer.

22. The optical device according to claim 21 wherein said computer control means includes visual display means for displaying images from light detected by said first light detector and wavelength dependent spectra from light detected by said spectrometer.

23. The optical device according to claim 21 wherein said second optical focusing means includes selectable light filter means for transmitting selected wavelengths of said light beam being imaged onto said first light detector said selectable light filter means includes a plurality of light filter in a motorized carousel, said motorized carousel being connected to said computer control means.

24. The device according to claim 21 wherein said second optical focusing means includes an optical fiber having a first end connected to said spectrometer and a second end located on a fiber mounting means located in said housing in a selected position so that said third light beam is focused onto said second end of said optical fiber.

25. The optical device according to claim 24 wherein said fiber mount means includes adjustment means for adjusting a position of the second end of the fiber in x, y and z directions.

26. The optical device according to claim 25 wherein said adjustment means is connected to an operator controlled actuator for operator controlled adjustment of the second end of said fiber within the image of the target area to enable the operator to record spectra from different locations on the image.

27. The optical device according to claim 26 wherein said operator controlled actuator is a hand-operated manipulator.

28. The device according to claim 21 wherein said positioning means is an articulating arm with said housing mounted on one end of said articulating arm and the other end of the articulating arm attached to a support frame, said articulating arm providing six degrees of freedom to said housing.

29. The device according to claim 21 wherein said first light detector is one of a charge coupled device (CCD) detector, an array detector, a pixilated detector, and a film detector.

30. A method for illuminating a target area and detecting electromagnetic radiation reflected or emitted by fluorescence from the target area, comprising the steps of;
shaping and directing a first light beam containing selected wavelengths along a first optical path and focusing said light beam onto a selected surface area of a target area; and
shaping and directing electromagnetic radiation reflected or emitted from said target area as a second light beam along a second optical path and focusing said electromagnetic radiation as an image onto a light detection means, wherein said first optical path and said second optical path share a common coaxial optical path section so that only the region of the target area where diffusely reflected light or light emitted by fluorescence are being detected is being illuminated by said first light beam; and
d) processing signals output from said light detection means and displaying the processed signals in a selected format.

31. The method according to claim 30 wherein said detection means includes a first light detector and a spectrometer, and including splitting said second light beam into a third light beam and directing said third light beam toward said spectrometer and a fourth light beam directed toward, and imaged onto, said first light detection and wherein the light signals focused onto the first light detector are displayed as an image of the target area from which light is diffusely reflected and/or emitted by fluorescence, and wherein light signals in the third light beam directed to said spectrometer are displayed as wavelength dependent spectra.

32. The method according to claim 31 wherein said step of splitting said second light beam into a fourth light beam includes filtering said fourth light beam through a selectable light filter for transmitting selected wavelengths of said light beam onto said first light detector means.

33. The method according to claim 31 wherein directing said third light beam to said spectrometer includes focusing said third light beam onto a second end of an optical fiber, said optical fiber having a first end connected to said spectrometer, said second end of the optical fiber being mounted on a fiber mounting means located in a selected position so that said third light beam is focused onto said second end of said optical fiber, including a step of adjustment of the second end of said fiber within the image of the target area to enable an operator to record spectra from different locations on the image of the target area.

34. The method according to claim 33 wherein said target area is biological tissue.

35. The method according to claim 34 wherein said fluorescence is autofluorescence of said tissue.

36. The method according to claim 33 wherein said target area is human tissue and said human tissue is dosed with a fluorophore compound which fluoresces at known wavelengths, and wherein said fluorescence is produced by said fluorophores concentrated in said human tissue.

37. The method according to claim 36 wherein said human tissue is a human brain of a patient undergoing in-situ diagnosis and treatment of diseased tissue utilizing light-induced-fluorescence imaging and point spectroscopy.

38. The method according to claim 37 wherein said step of processing signals output from said spectrometer and said first light detector includes displaying images and spectra produced therefrom in such a way that highlights optical differences between target tissue and/or surrounding tissue.

39. The method according to claim 37 wherein said light beam includes multispectral white-light, and including imaging of reflected light and fluorescence imaging of normal and tumorous brain tissue during neurosurgery.
